(19) United States
(12) Patent Application Publication
(10) Pub. No.: US 2025/0127546 A1
(43) Pub. Date: Apr. 24, 2025

(54) BONE FIXATION SYSTEM AND METHODS OF USE THEREOF

(71) Applicant: DePuy Synthes Products, Inc., Raynham, MA (US)

(72) Inventor: Dirk KERSTAN, West Chester, PA (US)

(21) Appl. No.: 18/493,139

(22) Filed: Oct. 24, 2023

(51) Int. Cl. A61B 17/80; A61B 17/00

(52) U.S. Cl. CPC A61B 17/8038; A61B 17/8061; A61B 2017/00867

(57) ABSTRACT

Bone fixation systems for use with fractured bones having at least one fragment are disclosed, along with kits and systems containing same, as well as methods of production and use thereof. The bone fixation systems include a bone anchor that matingly engages with an opening in a bone plate and that has a cannula extending through at least a portion of the bone anchor. The bone fixation systems also include at least one elongated member that is configured and adapted to extend between the interior of the bone anchor and an outer surface of the bone to embrace and/or anchor the at least one bone fragment in place against the remainder of the fractured bone.

### STATEMENT REGARDING FEDERALLY SPONSORED RESEARCH OR DEVELOPMENT

Not Applicable.

### CROSS REFERENCE TO RELATED APPLICATIONS/INCOPORATION BY REFERENCE STATEMENT

Not Applicable.

### BACKGROUND

The treatment of distal radius fractures requires a meticulous reconstruction of the joint surface, as well as stable internal fixation and early functional post-operative treatment. Extra-articular fractures require both the restoration of the volar tilt and radial length to reduce the possibility of displacement. Any malalignment may result in limitations of movement, changes of load distribution, and mid-carpal instability, as well as the increased risk of osteoarthritis in the radiocarpal joint. For example, intra-articular fractures with articular displacement of more than 2 mm in the radiocarpal joint inevitably result in osteoarthritis and functional impairment.

The distal radius and distal ulna form a three-column biomechanical construction: (1) the ulnar column is the distal ulna, the triangular fibrocartilage and the distal radio-ulnar joint; (2) the intermediate column is the medial part of the distal radius, with the lunate fossa and the sigmoid notch; and (3) the radial column is the lateral radius with the scaphoid fossa and the styloid process.

A dorsally displaced fracture of the distal radius indicates not only dorsiflexion in the sagittal plane, but also radial deviation in the frontal plane and supination in the transverse plane.

Following reduction, stabilization requires optimal fixation of the intermediate column as well as the radial column. In the case of a fractured distal ulna that compromises the distal radio-ulna joint, the ulnar column must be stabilized as well.

Treatment of volar ulnar corner fractures is currently the most central clinical problem of distal radius fracture fixation, because the patient comes back with a dislocated carpus if the fracture is not property addressed, and this condition is hard to address once it occurs. This fracture type occurs in up to 5% of distal radius cases that are treated operatively. Treatment of this fracture type requires fixation of small fragments of the distal radius' volar ulnar corner (also called “volar marginal fragments” of the distal radius or ulnar “bony avulsions” of the distal radius), and this fixation can be difficult to accomplish without compromising flexor tendons (FPL, FDP) running close to the fracture site. The solution needs to be as low profile as possible and easy to apply after a volar distal radius plate has been fixed (based on extensive VOC). Space is limited in this area for placement of a device, and access to this area is also limited.

Therefore, there is a need in the art for new and improved devices and methods of using same for fixation of distal radius fractures. It is to such devices, as well as methods of producing and using same, that the present disclosure is directed.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a perspective view of one non-limiting embodiment of a bone plate of a bone fixation system constructed in accordance with the present disclosure, wherein the bone plate is attached to a radius.

FIG. 2 is a perspective view of one non-limiting embodiment of a bone anchor of a bone fixation system constructed in accordance with the present disclosure.

FIG. 3 is a perspective view of one non-limiting embodiment of an elongated fixation member of a bone fixation system constructed in accordance with the present disclosure.

FIG. 4 is a perspective view of one non-limiting embodiment of a bone fixation system constructed in accordance with the present disclosure and comprising the elongated fixation member of FIG. 3.

FIG. 5 is a perspective view of another non-limiting embodiment of a bone fixation system constructed in accordance with the present disclosure and comprising at least two of the elongated fixation members of FIG. 3.

FIG. 6 is a perspective view of another non-limiting embodiment of an elongated fixation member of a bone fixation system constructed in accordance with the present disclosure.

FIG. 7 is a side view of yet another non-limiting embodiment of a bone fixation system constructed in accordance with the present disclosure and comprising the elongated fixation member of FIG. 6.

FIG. 8 is a perspective view of yet another non-limiting embodiment of an elongated fixation member of a bone fixation system constructed in accordance with the present disclosure.

FIG. 9 is a perspective view of yet another non-limiting embodiment of a bone fixation system constructed in accordance with the present disclosure and comprising the elongated fixation member of FIG. 8.

FIG. 10 is a cutaway view of the bone fixation system of FIG. 9, illustrating the bone anchor and the elongated fixation member.

FIG. 11 contains perspective views of elongated fixation members embracing (left) or embedding (right) a bone fragment against a remainder of the fractured bone.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the medical procedures and techniques of, surgery, anesthesia, wound healing, and infectious control described herein are those well-known and commonly used in the art. Standard techniques are used for infection diagnostic and therapeutic applications.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, systems, kits, and/or methods disclosed herein can be made and executed without undue experimentation in light of the present disclosure. While the articles, systems, kits, and/or methods have been described in terms of particular embodiments, it will be apparent to those of skill in the art that variations may be applied to the articles, systems, kits, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example (but not by way of limitation), when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with,” “coupled to,” and “connected to” include both direct association/coupling/connection of two elements to one another as well as indirect association/coupling/connection of two elements to one another. When two elements are indirectly associated/coupled/connected to one another, one or more intervening elements may be present therebetween. Non-limiting examples of intervening elements include washers, sleeves, fasteners, nuts, bolts, anchors, nails, inserts, rivets, bonding materials, and the like.

As used herein, the term “patient” or “subject” is meant to include all organisms, whether alive or dead, so long as the species has soft tissues and bones. For example, a system according to the inventive concepts disclosed herein may be applied to the bone of a living human, horse, cow, sheep, cat, dog, and the like.

Certain embodiments of the present disclosure are directed to a bone fixation system for a fractured bone, such as (but not limited to) a distal radial bone, and more particularly (but not by way of limitation) a volar ulnar surface of the radial bone. The bone fixation system is configured and adapted to embrace and/or anchor small bone fragments to the remaining radial bone and prevent the small bone fragments from escaping from the fixation system.

Treatment of volar ulnar corner fractures is currently the most central clinical problem of distal radius fracture fixation, because the patient comes back with a dislocated carpus if the fracture is not property addressed. This fracture type occurs in up to 5% of distal radius cases that are treated operatively. Treatment of this fracture type requires fixation of small fragments of the distal radius' volar ulnar corner (also called “volar marginal fragments” of the distal radius or ulnar “bony avulsions” of the distal radius), and this fixation can be difficult to accomplish without compromising flexor tendons (FPL, FDP) running close to fracture site. The solution needs to be as low profile as possible and easy to apply after a volar distal radius plate has been fixed (based on extensive VOC). Space is limited in this area for placement of a device, and access to this area is also limited.

One of the main functions of the devices/systems of the present disclosure is buttressing with occasionally a tension band component. Fragments resulting from a fracture in this region can be as small as 0.5 cm×1.5 cm; as such, the systems and methods of the present disclosure allow for holding the fragment(s) put rotationally stable without the need of drilling holes into the fragment and thus avoiding the risk of further splitting the fragment.

One non-limiting embodiment of the present disclosure includes implants that connect to the main fixation by “locking” into the cannula of a cannulated locking screw that was previously locked into the according distal ulnar hole of a volar distal radius plate. These implants feature a bridge of different lengths to accommodate different anatomies (sizes of the so-called “tear-drop”) and a distal end that can be either hooked around the fragment (embrace it) or dive into the fragment (to be embedded).

In certain non-limiting examples, the implant may be a “wire-type” implant that can be locked into the cannula by various mechanisms. For example (but not by way of limitation), the wire-type implant may be locked into the cannula by a roughened tail that features an S-curve to increase tension and friction within the cannula. As a non-limiting variation, both the wire tail and the hooks are connected to a button head that may feature a positive torx shape on its underside which mates with the T8 recess of the screw and this way establishes a rotationally stable construct.

Certain non-limiting embodiments of the present disclosure include a bone fixation system for a fractured bone. The bone fixation system includes a bone anchor configured and adapted to matingly engage with at least one opening in a bone plate and at least one elongated member that can be secured to the bone anchor while also embracing and/or otherwise anchoring at least one fractured fragment of the bone in place against a remainder of the bone.

The bone fixation systems of the present disclosure may be utilized with any bone that has at least one fractured fragment. In particular (but non-limiting) embodiments, the fractured bone may be the distal ulnar surface of a radius, and in particular (but not by way of limitation), the volar ulnar surface of a radius.

The bone anchor comprises a cannula extending through at least a portion of an interior of the bone anchor. In certain particular (but non-limiting) embodiments, a first end of the bone anchor is open, while a second end of the bone anchor is closed (and thus the cannula does not extend through the lower end of the bone anchor). In other particular (but non-limiting) embodiments, both the first and second ends of the bone anchor are open, and the cannula extends through the entirety of the bone anchor.

The term “elongated member” is used herein interchangeably with the terms “fixation member,” “elongated fixation member,” “retaining member,” and “implant.” The elongated member includes an anchor connector portion, at least one support portion, and at least one fragment connector portion. The anchor connector portion extends at an angle from a proximal end of the at least one support portion, and the at least one fragment connector portion extends at an angle from a distal end of the at least one support portion. The elongated member is configured and adapted to extend between the interior of the bone anchor and an outer surface of the bone. The anchor connector portion is configured to engage within the cannula in the bone anchor, and the at least one fragment connector portion is configured to embrace and/or otherwise anchor at least one fractured fragment of the bone in place against a remainder of the bone. In addition, when the second end of the bone anchor is open, the anchor connector portion of the elongated member may extend through the cannula of the bone anchor and into the bone.

In certain particular (but non-limiting) embodiments, the bone fixation system further includes the bone plate configured and adapted to be attached to a surface of the fractured bone. The bone plate includes at least one opening therein, wherein the bone anchor is configured and adapted to matingly engage with the at least one opening in the bone plate. In use, the bone plate is placed adjacent a surface of the fractured bone, and the bone anchor is disposed within the at least one opening in the bone plate and through the bone plate into the bone; then the anchor connector portion of the elongated member is disposed in and engages within the cannula in the bone anchor, and the at least one fragment connector portion of the elongated member extends beyond the bone plate and contacts at least one fractured fragment of the bone to embrace and/or otherwise anchor the at least one fractured fragment of the bone in place against a remainder of the bone.

Any bone plates known in the art or otherwise contemplated herein may be utilized in accordance with the present disclosure. One particular (but non-limiting) example of a bone plate that may be utilized in accordance with the present disclosure is a volar distal radius plate.

The anchor connector portion of the elongated member may be provided with any configuration and/or structure that allows the elongated member to function as described herein. For example (but not by way of limitation), the anchor connector portion of the elongated member may include a threaded pattern that mates and frictionally engages with an opposing threaded pattern present in the cannula of the bone anchor to secure the elongated member to the bone anchor. Other non-limiting examples of structures that may be present include roughened surfaces, helical threads, ramped ratchet structures, opposing screw threads, opposed ramped structures, pawls, and the like.

Alternatively (and/or in addition thereto), in certain particular (but non-limiting) embodiments, the anchor connector portion of the elongated member may include a non-linear configuration (such as, but not limited to, an S-shape) that is configured to engage a surface of the cannula within the bone anchor and thereby secure the elongated member within the bone anchor.

The at least one fragment connector portion of the elongated member may be provided with any configuration and/or structure that allows the elongated member to function as described herein. In addition, the at least one fragment connector portion of the elongated member may contact at least one fractured fragment of the bone in any configuration or manner that allows the at least one fragment connector portion to embrace and/or otherwise anchor the at least one fractured fragment of the bone in place against a remainder of the bone. For example (but not by way of limitation), the at least one fragment connector portion of the elongated member may be configured to frictionally engage, surround, compress, and/or otherwise embrace at least a portion of an outer surface of the at least one fractured fragment of the bone to thereby anchor the at least one fractured fragment of the bone in place against a remainder of the bone. Alternatively (and/or in addition thereto), the at least one fragment connector portion of the elongated member may be configured to extend into the at least one fractured fragment of the bone to thereby anchor the at least one fractured fragment of the bone in place against a remainder of the bone.

The at least one support portion of the elongated member may be provided with any structure and/or configuration that allows the elongated member to function as described herein. In certain particular (but non-limiting) embodiments, at least a portion (or all) of the support portion(s) of the elongated member has a linear configuration. Alternatively, at least a portion of the support portion(s) of the elongated member may have a non-linear configuration. For example (but not by way of limitation), in one non-limiting embodiment, the support portion(s) of the elongated member comprises at least three segments, wherein a first segment is adjacent the anchor connector portion, a second segment is disposed generally parallel to the first segment and adjacent the fragment connector portion, and a third segment extends between the first and second segments at a perpendicular angle whereby the second segment forms a step-off structure that is configured to be disposed beyond the bone plate and below an upper surface of the bone plate. In this manner, the step-off structure has a profile lower than the bone plate and allows the bone fixation system to assume as low a profile as possible and that closely approximates the physical structures present (i.e., the attachment of the bone plate to the larger section of bone).

In certain particular (but non-limiting) embodiments, the support portion(s) comprises particular structures that interact with the bone plate (as described above) and/or the bone anchor. For example (but not by way of limitation), the support portion(s) may be linear or curved, so that the elongated member assumes the configuration of a staple or a U-shaped wire. Alternatively, the support portion(s) of the elongated member may include a button that sits on the top of the bone anchor and from which at least one fragment connector portion extends at an angle. The button may be stationary or may allow for rotation of the at least one fragment connector portion of the elongated member to ensure adequate engagement of the bone fragment(s) with the at least one fragment connector portion of the elongated member.

The bone plates, bone anchors, and elongated members of the bone fixation devices/systems of the present disclosure may be formed of any materials that allow each of the components to function in accordance with the present disclosure.

In certain particular (but non-limiting) embodiments, at least a portion of each of the bone plates, bone anchors, and/or elongated members may be formed from a biocompatible material selected from the group consisting of stainless steel, titanium, a metal alloy (such as, but not limited to, a titanium alloy, a molybdenum metal alloy, or a cobalt base alloy), a compositive material, a polymeric material, a ceramic material, a shape memory material, and any combinations thereof.

In certain particular (but non-limiting) embodiments, at least a portion of the elongated member is formed of a rigid material.

In certain particular (but non-limiting) embodiments, at least a portion of the elongated member is formed of a contourable material. For example, but not by way of limitation, at least a portion of the elongated member may be formed of a deformable or transitionable material.

In certain particular (but non-limiting) embodiments, at least a portion of the elongated member may be manufactured from a shape memory material with superelastic or temperature dependent properties such that the elongated member transitions between a natural shape and an insertion shape. Non-limiting examples of shape memory materials that may be utilized in accordance with the present disclosure include shape memory alloys such as (but not limited to) a nickel-titanium alloy (e.g., nitinol).

For example (but not by way of limitation), at least a portion of the fragment connector portion(s) of the elongated member may be formed of a contourable material (e.g., a shape memory alloy such as (but not limited to) nitinol), and the contourable material compresses to exert a compressive force that embraces and frictionally engages the bone fragment so as to anchor the bone fragment to the remaining bone. In this manner, the elongated member may be referred to as a continuous compression implant.

Each of the portions of the elongated member may be formed of the same or different materials and may be provided with any thickness/diameter and/or lengths, so long as the elongated member is capable of functioning as described herein. The anchor connector portion of the elongated member may be longer than the fragment connector portion(s), or the fragment connector portion(s) may be longer than the anchor connector portion.

In a certain particular (but non-limiting) embodiment, the anchor connector portion of the elongated member is configured to extend through the bone anchor and into the bone. Also, as described herein above, the fragment connector portion(s) may also be configured to extend into at least one bone fragment. In these non-limiting instances, the anchor connector portion and/or the fragment connector portion(s) may be provided with any structures on the terminal ends thereof that allow the connector portion to engage and be anchored within the bone/bone fragment.

Certain non-limiting embodiments of the present disclosure are directed to a method of stabilizing a fractured bone having at least one fragment using any of the bone fixation systems disclosed or otherwise contemplated herein. In the method, any of the bone plates disclosed or otherwise contemplated herein is placed adjacent a surface of a fractured bone (such as, but not limited to, a distal ulnar surface of a radius, and in particular (but not by way of limitation), a volar ulnar surface of a radius). Any of the bone anchors disclosed or otherwise contemplated herein matingly engages with the at least one opening in the bone plate and is secured within the at least one opening in the bone plate and through the bone plate into the bone to secure the bone plate to the bone. Then, at least one of any of the elongated members disclosed or otherwise contemplated herein is secured to the bone anchor by disposing and engaging the anchor connector portion of the elongated member within the cannula in the bone anchor. The fragment connector portion(s) of the elongated member extends beyond the bone plate and contacts at least one fractured fragment of the bone to embrace and/or otherwise anchor the at least one fractured fragment of the bone in place against the remainder of the bone.

In certain particular (but non-limiting) embodiments, the fragment connector portion(s) of the elongated member frictionally engages, surrounds, compresses, and/or otherwise embraces at least a portion of an outer surface of the at least one fractured fragment of the bone to thereby anchor the at least one fractured fragment of the bone in place against a remainder of the bone. Alternatively (and/or in addition thereto), the fragment connector portion(s) of the elongated member extends into (and is thus embedded in) the at least one fractured fragment of the bone to thereby anchor the at least one fractured fragment of the bone in place against a remainder of the bone.

The at least one support portion of the elongated member may be provided with any structure(s) and/or configuration(s) that allow the anchor connector portions and the fragment connector portions of the elongated member to function as required herein. In certain particular (but non-limiting) embodiments, the support portion(s) is linear, curved, or non-linear, and may comprise a single or multiple segments. In certain particular (but non-limiting) embodiments, the support portion(s) of the elongated member comprises at least three segments, wherein a first segment is adjacent the anchor connector portion, a second segment is disposed generally parallel to the first segment and adjacent the fragment connector portion, and a third segment extends between the first and second segments at a perpendicular angle whereby the second segment forms a step-off structure. The step-off structure has a profile lower than the bone plate and is disposed beyond the bone plate and below an upper surface of the bone plate.

In a particular (but non-limiting) embodiment, the method of the present disclosure includes one or more of the following steps: installing a cannulated locking screw (i.e., a bone anchor) into a bone plate that has been positioned adjacent a radius, thereby securing the bone plate to the radius; selecting a properly sized implant (i.e., an elongated fixation member; this step may be performed with a probe with a drill guide); predrilling a fixation location with single or double drill guides; aligning both ends of the implant/elongated member; and inserting the implant/elongated member into the cannulated locking screw and optionally into the bone fragment (note that the direction of any hook on the second end of the implant/elongated member needs to be parallel to the direction of the cannulated screw/bone anchor in order to be insertable and/or to compress the fragment against the remaining bone).

In certain particular (but non-limiting) embodiments, the methods of the present disclosure may further include one or more steps of removing one or more elements of the bone fixation system after a period of time. In certain non-limiting embodiments, one or more elements of the bone fixation system may be provided with a feature or characteristic that aids in removal of the element from the radius. Non-limiting examples of features of characteristics include one or more perforations, threadings, grooves, graduations, roughened surfaces, and the like.

For each of the methods described herein, two or more steps may be performed simultaneously or wholly or partially sequentially. In addition, one or more of the steps may be performed immediately following a prior step, and/or a period of time may pass in between two or more steps.

Further, changes can be made in the order or sequence of steps, and one or more steps may be repeated, as desired, for any of the methods disclosed or otherwise contemplated herein.

Certain embodiments of the present disclosure are further directed to a kit that includes one or more of any of the elongated fixation members, bone plates, and/or bone anchors discussed in detail herein above or otherwise contemplated herein. In certain particular (but non-limiting) embodiments, the kit may include a plurality of one or more of the elongated fixation members, bone plates, and/or bone anchors, thereby allowing for selection of a properly sized member/plate/anchor from the kit.

The kit may also include at least one tool for use with inserting or removing any of the components present in the kit. For example, but not by way of limitation, the kit may also include forceps with “stepped” ends to mate with the bone anchor and/or elongated fixation member for supporting the element(s) during insertion and/or for engaging the element(s) during removal. Alternatively (and/or in addition thereto), the kit may include any other type of insertion or removal tool that engages any extension of overhang on one or more elements to assist in grabbing/supporting the element(s) during insertion and removal.

In addition, the kit may further contain one or more other component(s) or reagent(s) that may be utilized with the bone fixation systems in accordance with the present disclosure. For example (but not by way of limitation), the kit may include additional retaining elements (i.e., screws, pins, etc.) for placement in other openings in the bone plate to assist in anchoring the bone plate to the bone. The nature of these additional component(s)/reagent(s) will depend upon various factors, and identification thereof is well within the skill of one of ordinary skill in the art; therefore, no further description thereof is deemed necessary.

Also, the various components/reagents present in the kit may each be in separate containers/compartments, or various components/reagents can be combined in one or more containers/compartments. In addition, the kit may include a set of written instructions explaining how to use the kit. A kit of this nature can be used in any of the methods described or otherwise contemplated herein.

Turning now to the Drawings, FIGS. 1-3 illustrate various components of one non-limiting limiting embodiment of a bone fixation system 10 constructed in accordance with the present disclosure. A bone plate 12 is shown in FIG. 1 for securing to a fractured radius 14 of a subject. The bone plate 12 is configured and adapted to be attached to a surface 16 of the fractured radius 14. The bone plate 12 comprises various openings therein (a portion of which are indicated by reference numeral 18) through which various retaining elements (such as, but not limited to, pins, screws, staples, etc.) can be inserted to secure the bone plate 12 to the fractured radius 14. Bone plates having this design and configuration are well known in the art and commercially available.

In addition, the bone plate 12 comprises at least one opening 20 that is disposed adjacent a volar ulnar surface 22 of the radial bone 14. The at least one opening 20 is configured and adapted to receive a bone anchor and a portion of an elongated fixation member of the bone fixation system 10, as described in detail herein after.

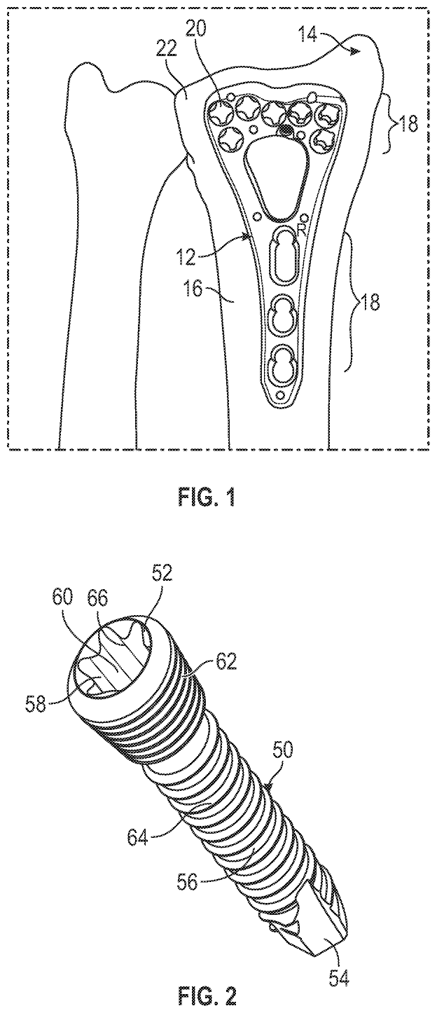

FIG. 2 illustrates one non-limiting embodiment of a bone anchor 50 that may be utilized in accordance with the bone fixation system 10. The bone anchor 50 includes an open first end 52, a second end 54 (which may be open or closed), an outer surface 56, and an interior surface 58 defining an interior retaining space/cannula 60 extending through at least a portion of the bone anchor 50.

One non-limiting embodiment of a structure that may function as a bone anchor in accordance with the present disclosure is a cannulated screw. Cannulated screws having this design and configuration are well known in the art and commercially available.

In certain non-limiting embodiments, the bone anchor 50 may further include a head 62 and a body 64. The head 62 extends from the first end 52 of the bone anchor 50 and may have an outer diameter that is larger than a diameter of the body 64. In addition, the head 62 may further include a recess 66 that is coextensive with the cannula 60 present in the body 64 of the bone anchor 50. In certain particular (but non-limiting) embodiments, the recess 66 has a diameter that is slightly larger than a diameter of the cannula 60.

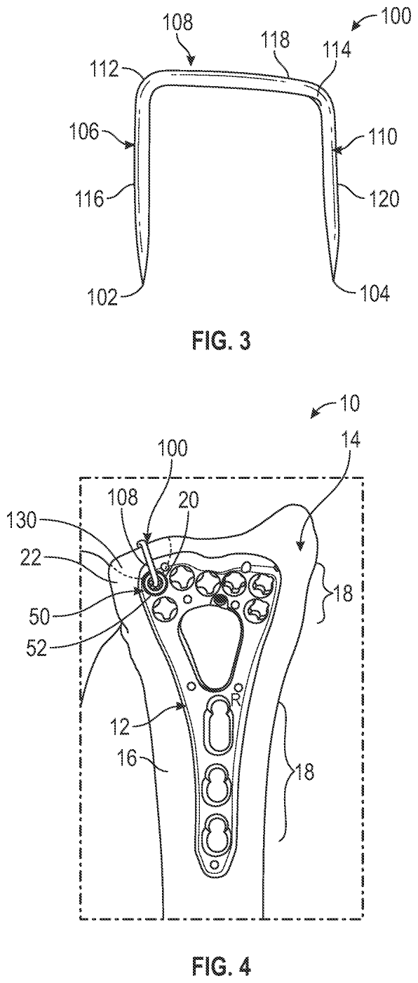

FIG. 3 illustrates one non-limiting embodiment of an elongated fixation or retaining member 100 that may be utilized in accordance with the bone fixation system 10. The elongated member 100 comprises a first end 102, a second end 104, and three segments: a first segment 106 (i.e., an anchor connector portion of the elongated member 100) that extends from the first end 102 of the elongated member; a second segment 108 (i.e., a support portion of the elongated member 100) that extends at an angle from the first segment 106; and a third segment 110 (i.e., a fragment connector portion of the elongated member 100) that extends at an angle from the second segment 108 to the second end 104 of the elongated member 100. The first segment 106 extends from a proximal end 112 of the second segment 108, and the third segment 110 extends from a distal end 114 of the second segment 108. The first segment 106 has an outer surface 116, the second segment 108 has an outer surface 118, and the third segment 110 has an outer surface 120.

The elongated member 100 is configured and adapted to extend between the interior of the bone anchor 50 and an outer surface of the bone 14, whereby the first segment 106 is configured to be frictionally engaged and secured within at least a portion of the cannula 60 in the bone anchor 50, and wherein the third segment 110 of the elongated member 100 is configured to embrace and/or otherwise anchor at least one bone fragment in place against a remainder of the fractured bone 14.

While the first and second ends 102 and 104 of the elongated member 100 are illustrated in FIG. 3 as being linear and pointed, it will be understood that the first and second ends 102 and 104 may be provided with any configuration that allows the elongated member 100 to function in accordance with the present disclosure. For example (but not by way of limitation), the first end 102 and/or the second end 104 of the elongated member 100 may be flat, blunt, angular, curved, grooved, stepped, and/or tapered.

The outer surface 116 of the first segment 106 of the elongated member 100 may be provided with any structure and/or configuration that allows at least a portion of the first segment 106 to engage the cannula 60 in the bone anchor 50 to thereby frictionally engage and secure the at least a portion of the first segment 106 within the bone anchor 50. For example (but not by way of limitation), at least a portion of the outer surface 116 may be threaded if the interior surface 58 of the bone anchor 50 is threaded, so that the threadings of the outer surface 116 and the interior surface 58 matingly engage one another and secure the elongated member 100 to the bone anchor 50. Alternatively, at least a portion of the outer surface 116 of the first segment 106 may be provided with other designs and configurations (e.g., smooth, roughened, tapered, graduated, etc.), so long as the first segment 106 is capable of matingly engaging the cannula 60 of the bone anchor 50.

The outer surface 118 of the second segment 108 of the elongated member 100 may be provided with any design and/or configuration that will allow the elongated member 100 to function in accordance with the present disclosure. For example, but not by way of limitation, the outer surface 118 of the second segment 108 may be smooth, tapered, graduated, threaded, etc. In addition, the second segment 108 may be provided with any structure and/or configuration that allows the first segment 106 to matingly engage the bone anchor 50 and the third segment 110 to embrace and/or otherwise anchor at least one bone fragment in place against a remainder of the fractured bone (as described in further detail herein). While the second segment 108 is depicted in FIG. 3 as having a linear configuration, this configuration is simply for purposes of illustration only; it will be understood that the second segment 108 may be curved or non-linear or any other configuration, so long as the elongated member is capable of functioning as described herein. Other elongated members having support portions (second segments) with non-linear configurations are disclosed further herein.

FIG. 4 illustrates an assembled bone fixation system 10 constructed in accordance with the present disclosure and that includes the components of FIGS. 1-3. The bone plate 12 is placed adjacent to the fractured radius 14 and secured thereto via one or more retaining elements (such as, but not limited to, pins, screws, staples, etc.) inserted through one or more openings 18. The bone anchor 50 is inserted through the opening 20 in the bone plate 12 and through the bone plate 12 into the radial bone 14. The first end 102 of the elongated fixation member 100 is then inserted into the cannula 60 in the bone anchor 50. The second segment 108 of the elongated member 100 extends at an angle from the first segment 106 of the elongated member 100, which is securingly engaged to the bone anchor 50. The third segment 110 of the elongated member 100 extends at an angle from the second segment 108 and embraces and/or otherwise anchors at least one bone fragment 130 in place against a remainder of the fractured radial bone 14. The first segment 106 of the elongated member 100 may rotate within the bone anchor 50 so that the second and third segments 108 and 110 attached thereto can be rotated to the appropriate location for interacting with the bone fragment(s) 130.

The third segment 110 of the elongated member 100 may interact with the bone fragment(s) 130 in any manner that allows the elongated member 100 to secure the bone fragment(s) 130 against the remainder of the fractured bone 14. For example (but not by way of limitation), the second end 104 of the elongated member 100 may extend into one or more bone fragment(s) 130. Alternatively, at least a portion of the third segment 110 of the elongated member may frictionally engage and exert a compressive force against the bone fragment(s) 130 to retain the bone fragment(s) 130 against the remainder of the fractured bone 14.

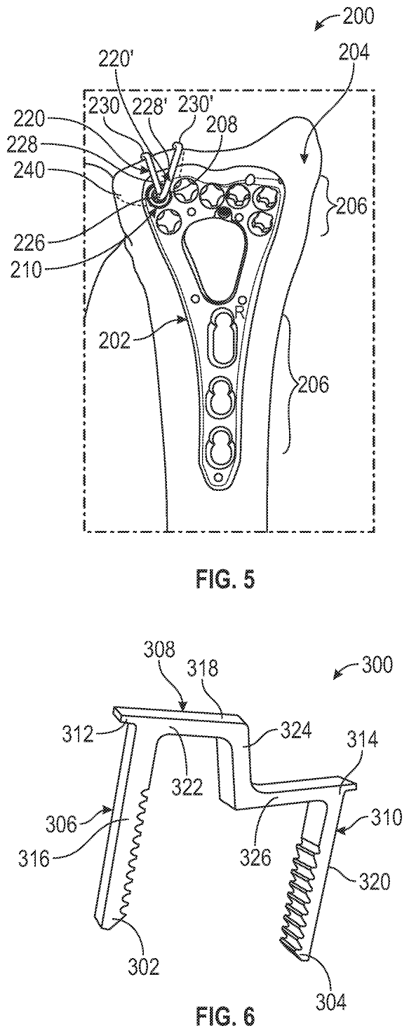

FIG. 5 illustrates another non-limiting embodiment of an assembled bone fixation system 200 constructed in accordance with the present disclosure. The assembled bone fixation system 200 is similar to the bone fixation system 10 of FIG. 4, except that the assembled bone fixation system 200 includes either two separate elongated members or a single elongated member that includes two second segments extending from a first segment, along with third segments extending from each of the two second segments.

In the bone fixation system 200, a bone plate 202 is placed adjacent to the fractured radius 204 and secured thereto via one or more retaining elements (such as, but not limited to, pins, screws, staples, etc.) inserted through one or more openings 206. A bone anchor 210 is inserted through the opening 208 in the bone plate 202 and through the bone plate 202 into the radial bone 204. At least one elongated fixation member 220 is then secured to the bone anchor 210 by inserting a first end of the elongated fixation member 220 into a cannula in the bone anchor 210, in the same manner as described above for the bone fixation system 10. A first segment 226 of the elongated member 220 is securingly engaged to the bone anchor 210, and at least one second segment 228 of the elongated member 220 extends at an angle from the first segment 226. Then a third segment 230 of the elongated member 220 extends at an angle from the second segment 228 and embraces and/or otherwise anchors at least one bone fragment 240 in place against a remainder of the fractured radial bone 204.

The bone fixation system 200 comprises either two separate elongated members 220 and 220′ or a single elongated member 220 that includes two second segments extending from a first segment, along with third segments extending from each of the two second segments.

In the non-limiting embodiment that comprises two separate elongated members 220 and 220′, both of the elongated members 220 and 220′ have first ends (not shown) that are both inserted into the cannula 212 in the bone anchor 210. Alternatively, the first segment 226 of the elongated member 220 may have a retaining space therein in which the first end of the elongated member 220′ is disposed. Then each of the elongated members 220 and 220′ has a third segment 230 and 230′ extending from the second segment 228 and 228′ thereof. In yet another embodiment, the elongated member 220 may comprise two second segments 228 and 228′, each with a third segment 230 and 230′ extending therefrom.

Whether present on a single elongated member or two separate elongated members, the two third segments 230 and 230′ each interact with one or more bone fragments 240 in any manner that allows the elongated member 220 (and 220′, if two separate elongated members) to secure the bone fragment(s) 240 against the remainder of the fractured bone 204. For example (but not by way of limitation), the second end(s) of the elongated member 220 (and/or 220′, if two separate elongated members) may extend into the bone fragment(s) 240, in the same manner as described above for the bone fixation system 10. Alternatively, at least a portion of the third segment(s) 230/230′ of the elongated member(s) 220 (and/or 220′, if two separate elongated members) may frictionally engage and exert a compressive force against the bone fragment(s) 240 to retain the bone fragment(s) 240 against the remainder of the fractured bone 204.

The first segment 226 (and the first segment 226′, if present) and/or second segments 228/228′ of the elongated member 220 (and/or 220′, if two separate elongated members) may rotate within the bone anchor 210 so that the second and third segments 228/228′ and 230/230′ can be rotated to the appropriate location for interacting with the bone fragment(s) 240. In a particular (but non-limiting) embodiment, the second and third segments 228 and 230 can be rotated independently of the second and third segments 228′ and 230′; alternatively, the second and third segments 228 and 230 are rotated with the second and third segments 228′ and 230′.

The third segments 230/230′ of the elongated member 220 (and/or 220′, if two separate elongated members) may interact with the bone fragment(s) 240 in any manner that allows the elongated member 220 (and/or 220′, if two separate elongated members) to secure the bone fragment(s) 240 against the remainder of the fractured bone 204. For example (but not by way of limitation), each of the second ends of the elongated member 220 (and/or 220′, if two separate elongated members) may extend into one or more bone fragment(s) 240. Alternatively, at least a portion of the third segments 230/230′ of the elongated member 220 (and/or 220′, if two separate elongated members) may frictionally engage and exert a compressive force against the bone fragment(s) 240 to retain the bone fragment(s) 240 against the remainder of the fractured bone 204.

While the elongated fixation members 100 and 220 of FIGS. 1-5 are illustrated herein as comprising three segments that each have a substantially linear configuration, it will be understood that each of the segments of any of the elongated fixation members constructed in accordance with the present disclosure may be provided with any designs and configurations, so long as the elongated fixation member is capable of functioning as described herein. For example (but not by way of limitation), FIG. 6 depicts another non-limiting example of an elongated fixation member 300 constructed in accordance with the present disclosure. The elongated member 300 comprises a first end 302, a second end 304, and three segments: a first segment 306 (i.e., an anchor connector portion of the elongated member 300) that extends from the first end 302 of the elongated member; a second segment 308 (i.e., a support portion of the elongated member 100) that extends at an angle from the first segment 306; and a third segment 310 (i.e., a fragment connector portion of the elongated member 300) that extends at an angle from the second segment 308 to the second end 304 of the elongated member 300. The first segment 306 extends from a proximal end 312 of the second segment 308, and the third segment 310 extends from a distal end 314 of the second segment 308. The first segment 306 has an outer surface 316, the second segment 308 has an outer surface 318, and the third segment 310 has an outer surface 320.

The second segment 308 is illustrated as having a non-linear configuration formed of a first section 322, a second section 324, and a third section 326, wherein the second section 324 extends at an angle from the first section 322, and the third section 326 extends at an angle from the second section 324. The three sections 322, 324, and 326 together form a step-off structure, wherein the second and third sections 324 and 326 are configured to be disposed beyond the bone plate and below an upper surface of the bone plate. In this manner, the step-off structure has a profile the same as or lower than the bone plate that allows the elongated element 300, and the bone fixation system as a whole, to assume as low a profile as possible and that closely approximates the physical structures present (i.e., the attachment of the bone plate to the larger section of bone).

The first segment 306 and the third segment 308 of the elongated member 300 are illustrated in FIG. 6 as being grooved or stepped, and the first and second ends 302 and 304 of the elongated member 300 are illustrated in FIG. 6 as being tapered. However, these configurations are for purposes of illustration only. It will be understood that the first and second ends 302 and 304 may be provided with any configuration that allows the elongated member 300 to function in accordance with the present disclosure. Likewise, the outer surfaces 316, 318, and/or 320 of the segments 306, 308, and/or 310, respectively, may be provided with any structure and/or configuration that allows the elongated member 300 to function as described herein.

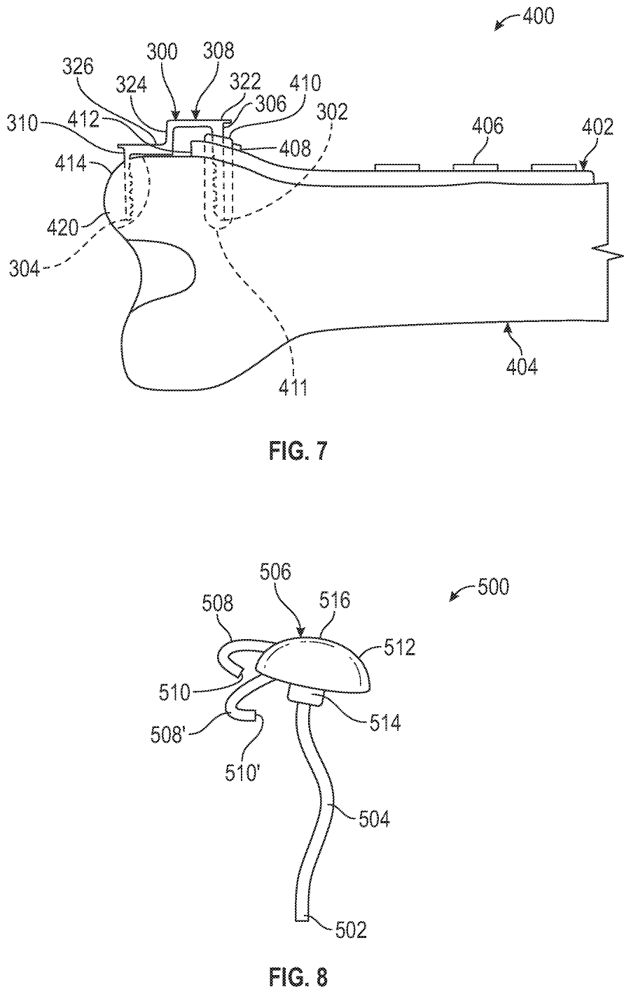

FIG. 7 illustrates an assembled bone fixation system 400 that comprises the elongated member 300 of FIG. 6. In the bone fixation system 400, a bone plate 402 is placed adjacent to a fractured radius 404 and secured thereto via one or more retaining elements (such as, but not limited to, pins, screws, staples, etc.) inserted through one or more openings 406. A bone anchor 410 is inserted through the opening 408 in the bone plate 402 and through the bone plate 402 into the radial bone 404. The elongated fixation member 300 of FIG. 6 is then secured to the bone anchor 410 by inserting the first end 302 of the elongated fixation member 300 into a cannula 411 in the bone anchor 410, in the same manner as described above for the bone fixation system 10. The first segment 306 of the elongated member 300 is securingly engaged to the bone anchor 410, and the second segment 308 of the elongated member 300 extends at an angle from the first segment 306. The second section 324 of the second segment 308 extends downwardly beyond a distal end 412 of the bone plate 402, and the third section 326 of the second segment 308 of the elongated member 300 is disposed substantially adjacent a surface 414 of the fractured radius 404. Then the third segment 310 of the elongated member 300 extends at an angle from the third section 326 of the second segment 308 and embraces and/or otherwise anchors at least one bone fragment 420 in place against a remainder of the fractured radial bone 404. For example, but not by way of limitation, the second end 304 of the elongated member 300 is illustrated in FIG. 7 as being embedded in the fragment 420; however, it will be understood that the second end 304 of the elongated member 300 need not be embedded in the fragment 420, but rather may embrace or otherwise compress the fragment 420 against the remainder of the bone 404.

FIG. 8 depicts another non-limiting example of an elongated fixation member 500 constructed in accordance with the present disclosure. The elongated member 500 comprises a first end 502; a first segment 504 (i.e., an anchor connector portion of the elongated member 500) that extends from the first end 502 of the elongated member 500; a second segment 506 (i.e., a support portion of the elongated member 500) that extends at an angle from the first segment 504; and a plurality of third segments 508 and 508′ (i.e., fragment connector portions of the elongated member 500) that each extends at an angle from the second segment 506 to second ends 510 and 510′ of the elongated member 500.

The first segment 504 is illustrated in FIG. 8 as having a curved configuration (such as, but not limited to, an S-tail or double S-tail configuration) that matingly engages with a cannula within a bone anchor constructed in accordance with the present disclosure and locks the elongated member 500 in place within the cannula of the bone anchor. However, the particular configuration shown in FIG. 8 is for purposes and example only, and the first segment 504 may be provided with any shape and/or configuration described or otherwise contemplated herein that will matingly engage with a cannula of any of the bone anchors disclosed or otherwise contemplated herein to secure the elongated member 500 within the bone anchor.

The second segment 506 is illustrated as comprising a button 512 (such as, but not limited to, a substantially rounded or substantially flat, low-profile button) that engages with an upper end of a bone anchor (such as, but not limited to, the upper end 52 of the bone anchor 50 of FIG. 2) and therefore further aids in securing the elongated member 500 to the bone anchor. For example (but not by way of limitation), the second segment 506 may be provided with a shape and/or configuration that engages the open first end 52 and/or the recess 66 in the head 62 of the bone anchor 50 of FIG. 2.

In certain non-limiting embodiments, the second segment 506 further includes a male connector 514 (such as, but not limited to, a torx drive head or T8 positive connector) on an underside of the button 512 that has a shape/configuration that mates with a shape/configuration of the recess in the head of any of the bone anchors disclosed or otherwise contemplated herein (such as, but not limited to, the recess 66 in the head 62 of the bone anchor 50 of FIG. 2) and that further assists in locking the elongated member 500 in place within the bone anchor. In addition, the button 512/connector 514 configuration of the second segment 506 allows for pressure to be applied to the elongated member 500 in securing the elongated member 500 in place within the head of the bone anchor. Alternatively, the button 512 may be provided with a recess that receives a portion of the head of the bone anchor for securing the elongated member 500 in place within the head of the bone anchor.

In certain non-limiting embodiments, an upper surface 516 of the button 512 may be provided with a shallow recess therein that has a shape/configuration that can matingly engage with an insertion and/or removal tool. In this manner, the shallow recess on the upper surface 516 of the button 512 can be engaged for removal of one or more parts of the bone fixation system.

The third segments 508/508′ are depicted in FIG. 8 as each having a curved or hooked configuration that extends out from a side of the button 512 and then downward at a curved or hooked angle for engaging one or more bone fragments. The third segments 508/508′ of the elongated member 500 may interact with the bone fragment(s) in any manner that allows the elongated member 500 to secure the bone fragment(s) against the remainder of the bone. For example (but not by way of limitation), each of the second ends of the elongated member 510 and 510′ may extend into one or more bone fragment(s). Alternatively, the curved or hooked configuration of each of the third segments 508/508′ of the elongated member 500 may surround at least a portion of and frictionally engage and exert a compressive force against the bone fragment(s) to retain the bone fragment(s) against the remainder of the bone.

The third segments 508/508′ may extend from the button 512 at any angle that allows the elongated member 500 to function in accordance with the present disclosure. For example, but not by way of limitation, an angle between the third segments 508/508′ may be about 10°,about 15°, about 20°, about 25°, about 30°, about 35°, about 40°, about 45°, about 50°, about 55°, about 60°, or higher, as well as a range formed between two of the above values (i.e., a range of from about 10° to about 60°, a range of from about 15° to about 55°, a range of from about 20° to about 40°, and the like).

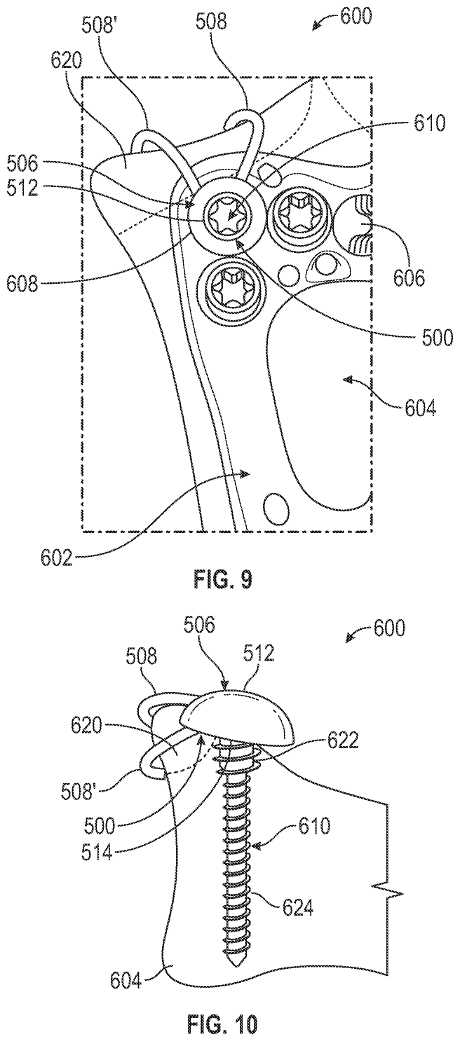

FIG. 9 illustrates an assembled bone fixation system 600 that comprises the elongated member 500. In the bone fixation system 600, a bone plate 602 is placed adjacent to a fractured radius 604 and secured thereto via one or more retaining elements (such as, but not limited to, pins, screws, staples, etc.) inserted through one or more openings 606. A bone anchor 610 is inserted through an opening 608 in the bone plate 602 and through the bone plate 602 into the radial bone 604. The elongated fixation member 500 of FIG. 8 is then secured to the bone anchor 610 by inserting the first end of the elongated fixation member 500 into the cannula in the bone anchor 610, in the same manner as described above for the bone fixation system 10. The first segment 504 of the elongated member 500 is securingly engaged to the bone anchor 610, and the second segment 506 of the elongated member 500 extends at an angle from the first segment 504.

The button 512 (and/or the male connector 514, if present) of the second segment 506 of the elongated member 500 engages with an upper end of a bone anchor 610 and aids in securing the elongated member 500 to the bone anchor 610. Then each of the third segments 508 and 508′ of the elongated member 500 extends at an angle from the second segment 506 and embraces and/or otherwise anchors at least one bone fragment 620 in place against a remainder of the fractured radial bone 604.

FIG. 10 is a cutaway view of the bone fixation system 600 of FIG. 9, illustrating the connection points between the bone anchor 610, the elongated fixation member 500, and the bone fragment(s) 620 and remainder of the fractured radial bone 604. As can be seen, the bone anchor 610 includes a head 622 and a body 624. The head 622 includes a recess that is coextensive with the cannula present in the body 624 of the bone anchor 610. In certain particular (but non-limiting) embodiments, the recess has a diameter that is slightly larger than a diameter of the cannula. The male connector 514 on the underside of the button 512 mates with the recess in the head 622 of the bone anchor 610 and further assists in locking the elongated member 500 in place within the bone anchor 610. In addition, the button 512/connector 514 configuration of the second segment 506 allows for pressure to be applied to the elongated member 500 in securing the elongated member 500 in place within the head 622 of the bone anchor 610.

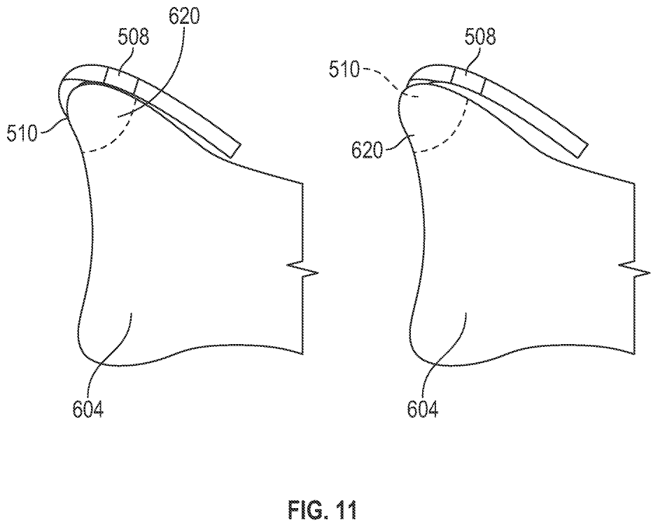

As discussed herein with reference to each of the Figures, any of the elongated fixation members described or otherwise contemplated herein may embrace one or more bone fragments and/or be embedded in the bone fragment(s) to frictionally engage and exert a compressive force against the bone fragment(s) and thereby retain the bone fragment(s) against the remainder of the fractured bone. For example (but not by way of limitation), the left panel of FIG. 11 illustrates the third segment 508 of the elongated fixation member 500 embracing the bone fragment 620 and retaining the bone fragment 620 against the remainder of the fractured radial bone 604. Alternatively, the right panel of FIG. 11 illustrates the third segment 508 of the elongated fixation member 500 embracing the bone fragment 620 while the second end 510 of the elongated fixation member 500 is embedded within the bone fragment 620 to thereby retain the bone fragment 620 against the remainder of the fractured radial bone 604.

While certain embodiments shown in the Drawings illustrate placement of the bone fixation systems of the present disclosure on a left arm, it will be understood that this illustration is for purposes of example only. The bone fixation systems of the present disclosure are equally applicable to use on a right arm.

While certain embodiments shown in the Drawings and described herein illustrate the devices, systems, kits, and methods of the present disclosure for use with a specific surface of a fractured radial bone, it will be understood that the systems and methods disclosed herein are not limited to use with a fractured radius. Therefore, the scope of the present disclosure explicitly includes devices, systems, kits, and methods that can be utilized with any radial fractures as well as any fractured bone containing bone fragments.

### NON-LIMITING ILLUSTRATIVE EMBODIMENTS

Illustrative embodiment 1. A bone fixation system for a fractured bone having at least one bone fragment, the bone fixation system comprising: a bone anchor configured and adapted to matingly engage with at least one opening in a bone plate, the bone anchor comprising a cannula extending through at least a portion of an interior of the bone anchor; and at least one elongated member comprising an anchor connector portion, at least one support portion, and at least one fragment connector portion, wherein the anchor connector portion extends from a proximal end of the at least one support portion and the at least one fragment connector portion extends from a distal end of the at least one support portion, wherein the elongated member is configured and adapted to extend between the interior of the bone anchor and an outer surface of the bone, whereby the anchor connector portion is configured to engage within the cannula in the bone anchor, and wherein the at least one fragment connector portion is configured to embrace and/or otherwise anchor the at least one bone fragment in place against a remainder of the fractured bone.

Illustrative embodiment 2. The system of Illustrative embodiment 1, wherein the at least one fragment connector portion of the elongated member is configured to frictionally engage, surround, and/or otherwise embrace at least a portion of an outer surface of the at least one fractured fragment of the bone to thereby anchor the at least one fractured fragment of the bone in place against a remainder of the bone.

Illustrative embodiment 3. The system of Illustrative embodiment 1, wherein the at least one fragment connector portion of the elongated member is configured to extend into the at least one fractured fragment of the bone to thereby anchor the at least one fractured fragment of the bone in place against a remainder of the bone.

Illustrative embodiment 4. The system of any of Illustrative embodiments 1-3, wherein the at least one support portion of the elongated member has a linear configuration.

Illustrative embodiment 5. The system of any of Illustrative embodiments 1-4, wherein at least a portion of the at least one support portion of the elongated member has a non-linear configuration.

Illustrative embodiment 6. The system of Illustrative embodiment 5, wherein the at least one support portion of the elongated member comprises at least three segments, wherein a first segment is adjacent the anchor connector portion, a second segment is disposed generally parallel to the first segment and adjacent the fragment connector portion, and a third segment extends between the first and second segments at a perpendicular angle whereby the second segment forms a step-off structure that is configured to be disposed beyond the bone plate and below an upper surface of the bone plate.

Illustrative embodiment 7. The system of any of Illustrative embodiments 1-6, wherein the at least one support portion of the elongated member comprises a button.

Illustrative embodiment 8. The system of any of Illustrative embodiments 1-7, wherein the anchor connector portion of the elongated member comprises a non-linear configuration configured to engage a surface of the cannula within the bone anchor and thereby secure the elongated member within the bone anchor.

Illustrative embodiment 8A. The system of any of Illustrative embodiments 1-8, at least a portion of the elongated member is formed of a rigid material.

Illustrative embodiment 8B. The system of any of Illustrative embodiments 1-8A, wherein at least a portion of the elongated member is formed of a contourable material.

Illustrative embodiment 9. The system of any of Illustrative embodiments 1-8B, wherein at least a portion of the elongated member is formed of a shape memory alloy.

Illustrative embodiment 10. The system of Illustrative embodiment 9, wherein the shape memory alloy is a nickel-titanium alloy.

Illustrative embodiment 11. The system of Illustrative embodiment 10, wherein the nickel-titanium alloy is nitinol.

Illustrative embodiment 12. The system of Illustrative embodiment 11, wherein at least a portion of the at least one fragment connector portion of the elongated member is formed of nitinol, wherein the nitinol compresses to embrace and anchor the bone fragment to the remaining bone.

Illustrative embodiment 13. The system of any of Illustrative embodiments 1-12, wherein the anchor connector portion of the elongated member is longer than the at least one fragment connector portion, or the at least one fragment connector portion is longer than the anchor connector portion.

Illustrative embodiment 14. The system of any of Illustrative embodiments 1-13, wherein the anchor connector portion of the elongated member is configured to extend through the bone anchor and into the bone.

Illustrative embodiment 15. A bone fixation system for a fractured bone having at least one fragment, the bone fixation system comprising: a bone plate configured and adapted to be attached to a surface of a fractured bone, the plate comprising at least one opening therein; a bone anchor configured and adapted to matingly engage with the at least one opening in the bone plate, the bone anchor comprising a cannula extending through at least a portion of an interior of the bone anchor; and at least one elongated member comprising an anchor connector portion, at least one support portion, and at least one fragment connector portion, wherein the anchor connector portion extends from a proximal end of the at least one support portion and the at least one fragment connector portion extends from a distal end of the at least one support portion, wherein the elongated member is configured and adapted to extend between the interior of the bone anchor and an outer surface of the bone; and wherein the bone plate is placed adjacent a surface of the fractured bone, the bone anchor is disposed within the at least one opening in the bone plate and through the bone plate into the bone, the anchor connector portion of the elongated member is disposed in and engages within the cannula in the bone anchor, and the at least one fragment connector portion of the elongated member extends beyond the bone plate and contacts at least one fractured fragment of the bone to embrace and/or otherwise anchor the at least one bone fragment in place against a remainder of the fractured bone.

Illustrative embodiment 16. The system of Illustrative embodiment 15, wherein the at least one fragment connector portion of the elongated member is configured to frictionally engage, surround, and/or otherwise embrace at least a portion of an outer surface of the at least one fractured fragment of the bone to thereby anchor the at least one fractured fragment of the bone in place against a remainder of the bone.

Illustrative embodiment 17. The system of Illustrative embodiment 15, wherein the at least one fragment connector portion of the elongated member is configured to extend into the at least one fractured fragment of the bone to thereby anchor the at least one fractured fragment of the bone in place against a remainder of the bone.

Illustrative embodiment 18. The system of any of Illustrative embodiments 15-17, wherein the at least one support portion of the elongated member has a linear configuration.

Illustrative embodiment 19. The system of any of Illustrative embodiments 15-18, wherein at least a portion of the at least one support portion of the elongated member has a non-linear configuration.

Illustrative embodiment 20. The system of Illustrative embodiment 19, wherein the at least one support portion of the elongated member comprises at least three segments, wherein a first segment is adjacent the anchor connector portion, a second segment is disposed generally parallel to the first segment and adjacent the fragment connector portion, and a third segment extends between the first and second segments at a perpendicular angle whereby the second segment forms a step-off structure that is configured to be disposed beyond the bone plate and below an upper surface of the bone plate.

Illustrative embodiment 21. The system of any of Illustrative embodiments 15-20, wherein the anchor connector portion of the elongated member comprises a non-linear configuration that engages a surface of the cannula within the bone anchor, thereby securing the elongated member within the bone anchor.

Illustrative embodiment 22. The system of any of Illustrative embodiments 15-21, wherein the at least one support portion of the elongated member comprises a button.

Illustrative embodiment 22A. The system of any of Illustrative embodiments 15-22, wherein at least a portion of the elongated member is formed of a rigid material.

Illustrative embodiment 22B. The system of any of Illustrative embodiments 15-22A, wherein at least a portion of the elongated member is formed of a contourable material.

Illustrative embodiment 23. The system of any of Illustrative embodiments 15-22B, wherein at least a portion of the elongated member is formed of a shape memory alloy.

Illustrative embodiment 24. The system of Illustrative embodiment 23, wherein the shape memory alloy is a nickel-titanium alloy.

Illustrative embodiment 25. The system of Illustrative embodiment 24, wherein the nickel-titanium alloy is nitinol.

Illustrative embodiment 26. The system of Illustrative embodiment 25, wherein at least a portion of the at least one fragment connector portion of the elongated member is formed of nitinol, wherein the nitinol compresses to embrace and anchor the at least one bone fragment to the remaining bone.

Illustrative embodiment 27. The system of any of Illustrative embodiments 15-26, wherein the anchor connector portion of the elongated member is longer than the at least one fragment connector portion, or wherein the at least one fragment connector portion is longer than the anchor connector portion.

Illustrative embodiment 28. The system of any of Illustrative embodiments 15-27, wherein the anchor connector portion of the elongated member extends beyond the bone anchor and into the bone.

Illustrative embodiment 29. The system of any of Illustrative embodiments 15-28, wherein the surface of the fractured bone is the distal ulnar surface.

Illustrative embodiment 30. The system of Illustrative embodiment 29, wherein the surface of the fractured bone is the volar ulnar surface.

Illustrative embodiment 30A. The system of any of illustrative embodiments 15-30, wherein the bone plate comprises a volar distal radius plate.

Illustrative embodiment 31. A method of stabilizing a fractured bone having at least one fragment, the method comprising the steps of: placing a bone plate adjacent a surface of a fractured bone, wherein the bone plate comprises at least one opening therein; securing a bone anchor within the at least one opening in the bone plate and through the bone plate into the bone to secure the bone plate to the bone, wherein the bone anchor matingly engages with the at least one opening in the bone plate, and wherein the bone anchor comprises a cannula extending through at least a portion an interior thereof; securing at least one elongated member to the bone anchor, the at least one elongated member comprising an anchor connector portion, at least one support portion, and at least one fragment connector portion, wherein the anchor connector portion extends from a proximal end of the at least one support portion and the at least one fragment connector portion extends from a distal end of the at least one support portion, wherein the anchor connector portion is disposed in and engages within the cannula in the bone anchor, and the at least one fragment connector portion of the elongated member extends beyond the bone plate and contacts at least one fractured fragment of the bone to embrace and/or otherwise anchor the at least one bone fragment in place against a remainder of the fractured bone.

Illustrative embodiment 32. The method of Illustrative embodiment 31, wherein the at least one fragment connector portion of the elongated member frictionally engages, surrounds, and/or otherwise embraces at least a portion of an outer surface of the at least one fractured fragment of the bone to thereby anchor the at least one fractured fragment of the bone in place against a remainder of the bone.

Illustrative embodiment 33. The system of Illustrative embodiment 31, wherein the at least one fragment connector portion of the elongated member extends into the at least one fractured fragment of the bone to thereby anchor the at least one fractured fragment of the bone in place against a remainder of the bone.

Illustrative embodiment 34. The method of any of Illustrative embodiments 31-33, wherein the at least one support portion of the elongated member has a linear configuration.

Illustrative embodiment 35. The method of any of Illustrative embodiments 31-34, wherein at least a portion of the at least one support portion of the elongated member has a non-linear configuration.

Illustrative embodiment 36. The method of Illustrative embodiment 35, wherein the at least one support portion of the elongated member comprises at least three segments, wherein a first segment is adjacent the anchor connector portion, a second segment is disposed generally parallel to the first segment and adjacent the fragment connector portion, and a third segment extends between the first and second segments at a perpendicular angle whereby the second segment forms a step-off structure that is configured to be disposed beyond the bone plate and below an upper surface of the bone plate.

Illustrative embodiment 37. The method of any of Illustrative embodiments 31-36, wherein the anchor connector portion of the elongated member comprises a non-linear configuration that engages a surface of the cannula within the bone anchor, thereby securing the elongated member within the bone anchor.

Illustrative embodiment 38. The method of any of Illustrative embodiments 31-37, wherein the at least one support portion of the elongated member comprises a button.

Illustrative embodiment 38A. The method of any of Illustrative embodiments 31-37, wherein at least a portion of the elongated member is formed of a rigid material.

Illustrative embodiment 38B. The method of any of Illustrative embodiments 31-38A, wherein at least a portion of the elongated member is formed of a contourable material.

Illustrative embodiment 39. The method of any of Illustrative embodiments 31-38B, wherein at least a portion of the elongated member is formed of a shape memory alloy.

Illustrative embodiment 40. The method of Illustrative embodiment 39, wherein the shape memory alloy is a nickel-titanium alloy.

Illustrative embodiment 41. The method of Illustrative embodiment 40, wherein the nickel-titanium alloy is nitinol.

Illustrative embodiment 42. The method of Illustrative embodiment 41, wherein at least a portion of the at least one fragment connector portion of the elongated member is formed of nitinol, wherein the nitinol compresses to embrace and anchor the at least one bone fragment to the remaining bone.

Illustrative embodiment 43. The method of any of Illustrative embodiments 31-42, wherein the anchor connector portion of the elongated member is longer than the at least one fragment connector portion, or wherein the at least one fragment connector portion is longer than the anchor connector portion.

Illustrative embodiment 44. The method of any of Illustrative embodiments 31-43, wherein the anchor connector portion of the elongated member extends through the bone anchor and into the bone.

Illustrative embodiment 45. The method of any of Illustrative embodiments 31-44, wherein the surface of the fractured bone is the distal ulnar surface.

Illustrative embodiment 46. The method of Illustrative embodiment 45, wherein the surface of the fractured bone is the volar ulnar surface.

Illustrative embodiment 47. The method of any of Illustrative embodiments 31-46,wherein the bone plate comprises a volar distal radius plate.

Thus, in accordance with the present disclosure, there have been provided devices, assemblies, and kits, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

1. A bone fixation system for a fractured bone having at least one bone fragment, the bone fixation system comprising:
a bone anchor configured and adapted to matingly engage with at least one opening in a bone plate, the bone anchor comprising a cannula extending through at least a portion of an interior of the bone anchor; and
at least one elongated member comprising an anchor connector portion, at least one support portion, and at least one fragment connector portion, wherein the anchor connector portion extends from a proximal end of the at least one support portion and the at least one fragment connector portion extends from a distal end of the at least one support portion, wherein the elongated member is configured and adapted to extend between the interior of the bone anchor and an outer surface of the bone, whereby the anchor connector portion is configured to engage within the cannula in the bone anchor, and wherein the at least one fragment connector portion is configured to anchor the at least one bone fragment in place against a remainder of the fractured bone.

2. The system of claim 1, wherein the at least one support portion of the elongated member has a linear configuration.

3. The system of claim 1, wherein the at least one support portion of the elongated member has a non-linear configuration.

4. The system of claim 1, wherein the at least one support portion of the elongated member comprises a button.

5. The system of claim 1, wherein the anchor connector portion of the elongated member comprises a non-linear configuration configured to engage a surface of the cannula within the bone anchor and thereby secure the elongated member within the bone anchor.

6. The system of claim 1, wherein at least a portion of the elongated member is formed of a rigid material.

7. The system of claim 1, wherein at least a portion of the elongated member is formed of a contourable material.

8. The system of claim 7, wherein at least a portion of the elongated member is formed of a shape memory alloy.

9. The system of claim 8, wherein at least a portion of the at least one fragment connector portion of the elongated member is formed of nitinol, wherein the nitinol compresses to embrace and anchor the bone fragment to the remaining bone.

10. The system of claim 1, wherein the anchor connector portion of the elongated member is configured to extend through the bone anchor and into the bone.

11. A bone fixation system for a fractured bone having at least one fragment, the bone fixation system comprising:
a bone plate configured and adapted to be attached to a surface of a fractured bone, the plate comprising at least one opening therein;
a bone anchor configured and adapted to matingly engage with the at least one opening in the bone plate, the bone anchor comprising a cannula extending through at least a portion of an interior of the bone anchor; and
at least one elongated member comprising an anchor connector portion, at least one support portion, and at least one fragment connector portion, wherein the anchor connector portion extends from a proximal end of the at least one support portion and the at least one fragment connector portion extends from a distal end of the at least one support portion, wherein the elongated member is configured and adapted to extend between the interior of the bone anchor and an outer surface of the bone; and
wherein the bone plate is placed adjacent a surface of the fractured bone, the bone anchor is disposed within the at least one opening in the bone plate and through the bone plate into the bone, the anchor connector portion of the elongated member is disposed in and engages within the cannula in the bone anchor, and the at least one fragment connector portion of the elongated member extends beyond the bone plate and contacts at least one fractured fragment of the bone to anchor the at least one bone fragment in place against a remainder of the fractured bone.

12. The system of claim 11, wherein the at least one support portion of the elongated member has a linear configuration.

13. The system of claim 11, wherein the at least one support portion of the elongated member has a non-linear configuration.

14. The system of claim 11, wherein the anchor connector portion of the elongated member comprises a non-linear configuration that engages a surface of the cannula within the bone anchor, thereby securing the elongated member within the bone anchor.

15. The system of claim 11, wherein the at least one support portion of the elongated member comprises a button.

16. The system of claim 11, wherein at least a portion of the elongated member is formed of a rigid material.

17. The system of claim 11, wherein at least a portion of the elongated member is formed of a contourable material.

18. The system of claim 17, wherein at least a portion of the elongated member is formed of a shape memory alloy.

19. The system of claim 18, wherein at least a portion of the at least one fragment connector portion of the elongated member is formed of nitinol, wherein the nitinol compresses to embrace and anchor the at least one bone fragment to the remaining bone.

20. The system of claim 11, wherein the anchor connector portion of the elongated member extends beyond the bone anchor and into the bone.

21. The system of claim 11, wherein the bone plate is configured and adapted to be attached to a distal ulnar surface of a radius.

22. The system of claim 11, wherein the bone plate is configured and adapted to be attached to a volar ulnar surface of a radius.

23-34. (canceled)
